(19) United States
(12) Patent Application Publication
(10) Pub. No.: US 2022/0265659 A1
(43) Pub. Date: Aug. 25, 2022

(54) METHODS AND COMPOSITIONS FOR TREATING PAX6-DEFICIENCY RELATED DISEASE

(71) Applicants: INSERM (INSTITUT NATIONAL DE LA SANTÉ ET DE AL RECHERCHE MÉDICALE), Paris (FR); UNIVERSITÉ DE PARIS, Paris (FR); ASSISTANCE PUBLIQUE-HÔPITAUX DE PARIS (APHP), Paris (FR); SORBONNE UNIVERSITÉ, Paris (FR); RAMOT AT TEL-AVIV UNIVERSITY LTD, Tel Aviv (IL)

(72) Inventors: Daniel ABERDAM, Paris (FR); Dominique BREMONE-GIGNAC, Paris (FR); Lauriane ROUX, Pantin (FR); Ruth ASHERY PADAN, Tel Aviv (IL); Eddy PICHINUK, Tel Aviv (IL); Orly DOROT, Tel Aviv (IL); Keren KIPNIS, Tel Aviv (IL)

(21) Appl. No.: 17/637,682

(22) PCT Filed: Aug. 31, 2020
(86) PCT No.: PCT/EP2020/074241

§ 371 Date: Feb. 23, 2022

(30) Foreign Application Priority Data
Sep. 2, 2019 (EP) 19306060.5

(51) Int. Cl. A61K 31/519; A61K 31/4745; A61K 31/381

(52) U.S. Cl. CPC A61K 31/519; A61K 31/4745; A61K 31/381

(57) ABSTRACT

The present invention relates to an agent for increasing PAX6 gene and/or protein expression in a subject in need thereof. The insertion of a nonsense mutation found in patients allowed inventors to identify altered gene expression of PAX6 target genes, enhanced cell adhesion and reduced cell migration and proliferation. Remarkably, these parameters were rescued by recombinant PAX6 protein. Using this unique cellular model in which they inserted multiple copies of PAX6 responsive elements and tdTomato, they screened a FDA-approved library and identified in a first hit, 3 small compounds that enhance production of PAX6 protein while two of them did not modulate PAX6 gene expression. More particularly, they observed that the small compounds efficiently rescue in vitro migration of mutated limbal cells. Moreover, in the art, it is known that a decrease of PAX6 leads to an induction of diabetes. Small compounds identified by the inventors are suitable to treat PAX6-related deficiencies diseases such as aniridia and diabetes.

### FIELD OF THE INVENTION

The invention is in the field of medicine. More particularly, the invention relates to methods and compositions for treating PAX6-related deficiency diseases in a subject in need thereof.

### BACKGROUND OF THE INVENTION

The cornea, which is the outermost tissue of the eye, serves as a barrier against external insults and plays an optic role as a major light focusing structure. The corneal epithelium rests on a fibroblast stromal layer and is renewed by limbal stem cells (LSCs) that are located at the corneal/conjunctival transition zone, known as the limbus (1).

Loss of corneal transparency is frequently caused by aniridia, a bilateral panocular rare disease with an incidence of 1:64,000-1:96,000 (2). This mainly inherited disease (2:3 of cases) is caused by heterozygous mutations in PAX6, the master gene of eye development. Most of them are nonsense mutations, which generate premature termination codons that abolish the production of PAX6 protein due to nonsense-mediated mRNA decay and thus result in haploinsufficiency (2). The reduced level of PAX6 protein leads to several ocular deficits including iris hypoplasia, cataracts, fovea and optic nerve dysplasia, glaucoma and most prominently, the severe corneal phenotype of aniridia-related keratopathy (ARK) characterized by conjunctival cell ingrowth, corneal neo-vascularization and opacity with eventual visual loss (3). Complete ablation of the Pax6 gene in different organisms leads to a failure in eye formation while Pax6 heterozygous mice display a small eye phenotype and thus does not fully recapitulate human disease (4-7). Despite the documentation of corneal phenotypes in Pax6 mutants, the gene network regulated by PAX6 in healthy cornea remains poorly known (8,9). It has been speculated that the limbal niche is dysfunctional in eyes of Pax6 heterozygous mice (4).

In addition to ocular functions, Pax6 has been shown to regulate insulin and glucagon production by pancreatic cells (17) and maintain cell identity (18). Pax6+/− mice display abnormal metabolic homeostasis and reduced insulin secretion (19). Accordingly, diabetes has been associated with aniridia patients (20,21). Thus, there is current need to identify compounds/drugs to ameliorate and/or prevent ARK due to PAX6 haploinsufficiency, LSCD but also to modulate insulin production in PAX6-related diseases.

### SUMMARY OF THE INVENTION

The invention relates to an agent for increasing PAX6 gene and/or protein expression in a subject in need thereof. In particular, the invention is defined by claims.

### DETAILED DESCRIPTION OF THE INVENTION

Recently, inventors have performed a cellular model of ARK in vitro by using Crispr/cas9 on limbal stem cells (mut-LSC) (3). The insertion of a nonsense mutation found in patients allowed inventors to identify altered gene expression of PAX6 target genes, enhanced cell adhesion and reduced cell migration and proliferation. Remarkably, these parameters were rescued by recombinant PAX6 protein (3). Using this unique cellular model in which they inserted multiple copies of PAX6 responsive elements and tdTomato (TRE-tomato mut-LSC), they screened a FDA-approved library and identified in a first hit, 3 small compounds that enhance production of PAX6 protein and expression of PAX6-target genes. More particularly, they observed that the small compounds efficiently rescue in vitro migration of mutated limbal stem cells (mut-LSCs). Small compounds as identified are able to target all mutation/deletion in PAX6 which lead to a reduction of PAX6 level (PAX6 haploinsufficiency). Moreover, in the art, it is known that a decrease of PAX6 leads to an induction of diabetes. Accordingly, small compounds identified by the inventors are suitable to treat PAX6-related deficiency diseases such as aniridia and diabetes.

Method for Treating PAX6-Related Deficiencies Diseases

Accordingly, in a first aspect, the invention relates to an agent for use for increasing PAX6 gene and/or protein expression in a subject in need thereof.

In a particular embodiment, the invention relates to an agent for use for increasing PAX6 gene and/or protein expression for use in the treatment of PAX6-related deficiency diseases.

In particular, the invention relates to a method for treating a subject suffering from PAX6-related deficiency diseases comprising a step of administering to said subject a therapeutically effective amount of an agent which increases PAX6 gene and/or protein expression in a subject in need thereof.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “PAX6”, also known as aniridia type II protein (AN2) or oculorhombin relates to Paired box protein PAX-6. It is a protein that in humans is encoded by the PAX6 gene. PAX 6 is considered as a transcription factor PAX6 and is expressed in most ocular tissues during embryogenesis and in general this wide expression pattern is maintained through life. The naturally occurring human PAX6 gene has a nucleotide sequence as shown in Genbank Accession numbers NM_000280, NM_001127612, NM_001258462, NM_001258463 and NM_001258464. The naturally occurring human PAX6 protein has an aminoacid sequence as shown in Genbank Accession numbers NP_000271, NP_001121084, NP_001245391, NP_001245392 and NP_001245393.

As used herein, the term “PAX6-related deficiency diseases” relates to diseases which have at least one deficiency in PAX6 gene and/or protein. Typically, the deficiency in PAX-6 gene and/or protein level leads to decrease in brain size, brain structure abnormality leading to Autism, lack of iris formation or a thin cornea. Knockout experiments produced eyeless phenotypes reinforcing indications of the gene's role in eye development.

In a particular embodiment, the PAX6-related deficiency diseases are selected in the group consisting of but not limited to: PAX6 protein displays important functions in the development of the eye, nose, central nervous system and pancreas. PAX6 missense mutations in the HD domain may be associated with neurological or cognitive deficits. Mutations in PAX6 gene underlie also both autosomal dominant keratitis (OMIM: 148190) (eye disorder characterized by corneal opacification and vascularization and by foveal hypoplasia) and Peters anomaly (OMIM: 604229 612968) both implicated PAX6 broadly in human anterior segment malformations. Decreased auditory interhemispheric transfer function and defective central auditory tests suggested a role for the PAX6 gene in the neurodevelopment of higher-order auditory processing (Bamiou, D.-E., Musiek, F. E., Sisodiya, S. M., Free, S. L., Mitchell, T. N., Davies, R. A. Defective auditory interhemispheric transfer in a patient with a PAX6 mutation. Neurology 62: 489-490, 2004). Other genetic disorders that have been reported with limbal stem cell deficiency (LSCD) include ectrodactylyl-ectodermal-dysplasia-clefting syndrome (Hatch K M, Dana R. The structure and function of the limbal stem cell and the disease states associated with limbal stem cell deficiency. International ophthalmology clinics 2009; 49(1):43-52), keratitis-ichthyosis-deafness (KID) Syndrome (Di Iorio E, Kaye S B, Ponzin D, et al. Limbal stem cell deficiency and ocular phenotype in ectrodactyly-ectodermal dysplasia-clefting syndrome caused by p63 mutations. Ophthalmology 2012; 119(1):74-83), Xeroderma Pigmentosum (Messmer E M, Kenyon K R, Rittinger O, Janecke A R, Kampik A. Ocular manifestations of keratitis-ichthyosis-deafness (KID) syndrome. Ophthalmology 2005; 112(2):e1-6), Dominantly Inherited Keratitis (Fernandes M, Sangwan V S, Vemuganti G K. Limbal stem cell deficiency and xeroderma pigmentosum: a case report. Eye 2004; 18(7):741-3), Turner Syndrome (Lim P, Fuchsluger T A, Jurkunas U V. Limbal stem cell deficiency and corneal neovascularization. Seminars in ophthalmology 2009; 24(3):139-48), congenital glaucoma and Dyskeratosis Congenita (Aslan D, Ozdek S, Camurdan O, Bideci A, Cinaz P. Dyskeratosis congenita with corneal limbal insufficiency. Pediatric blood & cancer 2009; 53(1):95-7).

In a particular embodiment, the PAX6-related deficiency diseases are aniridia and/or diabetes.

In a particular embodiment, the PAX6-related deficiency disease is aniridia.

As used herein, the term “aniridia” refers to a bilateral panocular rare disease with an incidence of 1:64,000-1:96,000. The major diagnostic feature is congenital absence or hypoplasia of the iris; foveal hypoplasia with reduced visual acuity is almost always present and is usually associated with nystagmus. Aniridia complications result in progressive sight-threatening complications include keratopathy, cataract and glaucoma. Aniridia is either isolated without systemic involvement or a part of a syndrome. Isolated aniridia mainly exhibits autosomal dominant inheritance. The Wilms tumor, aniridia, genital abnormalities, and mental retardation (WAGR) syndrome is a representative syndromic form of aniridia, and is caused by contiguous gene deletion of both PAX6 and the adjacent Wilms tumor 1 (WT1) gene. Aniridia subjects may have other sensory deficits including reduced olfaction and hearing difficulties. Newborns with sporadic aniridia are at greatly increased risk of the paediatric nephroblastoma Wilms tumour if they have a deletion encompassing PAX6 and the nearby Wilms tumour predisposition gene (WT1 gene). Such deletions may manifest as Wilms tumour-aniridia-genital anomalies-retardation (WAGR) syndrome. Missense mutations in the PAX6 paired domain often cause atypical phenotypes ranging from mild iris hypoplasia to Peters anomaly and microphthalmia.

In another embodiment, the PAX6-related deficiency disease is diabetes.

As used herein, the term “diabetes” has its general meaning in the art and refers to the chronic disease characterized by relative or absolute deficiency of insulin that results in hypercglycemia. The term “diabetes” is thus intended to include those individuals with hyperglycemia, including chronic hyperglycemia, hyperinsulinemia, impaired glucose homeostasis or tolerance, and insulin resistance.

In a particular embodiment, the PAX6-related deficiency disease is Type 1 diabetes mellitus.

As used herein, the term “Type 1 diabetes mellitus” or “T1D” has its general meaning in the art and refers to an autoimmune disorder than leads to destruction of the insulin producing beta cells of the pancreas leading to hyperglycemia.

In a particular embodiment, the PAX6-related deficiency disease is type 2 diabetes.

As used herein, the term “type 2 diabetes” or “non-insulin dependent diabetes mellitus (NIDDM)” has its general meaning in the art. Type 2 diabetes often occurs when levels of insulin are normal or even elevated and appears to result from the inability of tissues to respond appropriately to insulin. Most of the type 2 diabetics are obese. As used herein the term “obesity” refers to a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m2 to less than 25 kg/m².

As used herein, the term “subject” refers to any mammals, such as a rodent, a feline, a canine, and a primate. Particularly, in the present invention, the subject is a human afflicted with or susceptible to be afflicted with aniridia.

In particular embodiment, the subject is a human afflicted with or susceptible to be afflicted with isolated aniridia.

In a particular embodiment, the subject is a human afflicted with or susceptible to be afflicted with keratopathy.

In particular embodiment, the subject is a human afflicted with or susceptible to be afflicted with cataract.

In particular embodiment, the subject is a human afflicted with or susceptible to be afflicted with glaucoma.

In particular embodiment, the subject is a human afflicted with or susceptible to be afflicted with diabetes.

In particular embodiment, the subject is a human afflicted with or susceptible to be afflicted with Type 1 diabetes mellitus.

In particular embodiment, the subject is a human afflicted with or susceptible to be afflicted with type 2 diabetes.

As used herein, the term “agent for use for increasing PAX6 gene and/or protein expression” denotes an agent which can increase or restore the PAX6 gene and/or protein expression. More particularly, the agent is able to rescue PAX6 gene expression at the RNA level (e.g. topotecan), and thus to enhance the transcription of PAX6.

In another embodiment, the agent is able to increase or restore PAX6 protein expression (e.g. duloxetine or ritanserine) and thus stabilizing PAX6 protein.

In a further embodiment, the agent for increasing PAX6 gene and/or protein expression is suitable for treating a subject suffering from PAX6-related deficiencies diseases.

As used herein, the term “agent for use for increasing PAX6 gene and/or protein expression” refers to a natural or synthetic compound which binds and activates PAX6 for initiating a pathway signalling and further biological processes. In the context of the invention, the agent enhances the transcription of PAX6 and increases proliferation and migration of cells (limbal, beta cells etc) in a subject.

Typically, the agent for use for increasing PAX6 protein gene and/or expression is PAX6, an aptamer, a small molecule, an antibody, a peptide, a polypeptide peptidomimetic or glycomimetic.

In a particular embodiment, the agent for use for increasing PAX6 protein expression is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the agent for use for increasing PAX6 gene and/or protein expression is a peptide.

In a particular embodiment, the agent for use for increasing PAX6 gene and/or protein expression is a polypeptide.

The term “polypeptide” refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups). In a particular embodiment, the polypeptide is a functional equivalent fragment of PAX6.

In a particular embodiment, the polypeptide is a peptidomimetic. As used herein, the term “peptidomimetic” refers to a polypeptide designed to mimic a peptide. The polypeptide may be produced by any suitable means, as will be apparent to those of skill in the art. In order to produce sufficient amounts of PAX6 or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known. When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein.

In a particular embodiment, the agent for use for increasing PAX6 gene and/or protein expression is an isolated, synthetic or recombinant PAX6 protein.

In some embodiments, the agent for use for increasing PAX6 gene and/or protein expression of the invention is selected from the group consisting of an isolated, synthetic or recombinant nucleic acid encoding for PAX6 protein, a nucleic acid sequence encoding for the fusion protein, a nucleic acid encoding a fragment of a PAX6 protein, a nucleic acid encoding a fragment of a peptide, a cell expressing PAX6 protein, and agent inducing PAX6 gene expression and their combinations.

As used herein, a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

These nucleic acid sequences can be obtained by conventional methods well known to those skilled in the art. Typically, said nucleic acid is a DNA or RNA molecule, which may be included in a suitable vector, such as a plasmid, cosmid, episome, artificial chromosome, phage or viral vector. So, a further object of the present invention relates to a vector and an expression cassette in which a nucleic acid molecule encoding for a polypeptide or a fusion protein of the invention is associated with suitable elements for controlling transcription (in particular promoter, enhancer and, optionally, terminator) and, optionally translation, and also the recombinant vectors into which a nucleic acid molecule in accordance with the invention is inserted. These recombinant vectors may, for example, be cloning vectors, or expression vectors.

As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Any expression vector for animal cell can be used. Examples of suitable vectors include pAGE107 (Miyaji et al., 1990), pAGE103 (Mizukami and Itoh, 1987), pHSG274 (Brady et al., 1984), pKCR (O'Hare et al., 1981), pSG1 beta d2-4 (Miyaji et al., 1990) and the like.

Other examples of plasmids include replicating plasmids comprising an origin of replication, or integrative plasmids, such as for instance pUC, pcDNA, pBR, and the like.

Other examples of viral vectors include adenoviral, lentiviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

Examples of promoters and enhancers used in the expression vector for animal cell include early promoter and enhancer of SV40 (Mizukami and Itoh, 1987), LTR promoter and enhancer of Moloney mouse leukemia virus (Kuwana et al., 1987), promoter (Mason et al., 1985) and enhancer (Gillies et al., 1983) of immunoglobulin H chain and the like.

A further aspect of the invention relates to a host cell comprising a nucleic acid molecule encoding for a peptide or a fusion protein according to the invention or a vector according to the invention. In particular, a subject of the present invention is a prokaryotic or eukaryotic host cell genetically transformed with at least one nucleic acid molecule or vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

In a particular embodiment, for expressing and producing proteins, peptides or fusion proteins of the invention, prokaryotic cells, in particular E. coli cells, will be chosen. Actually, according to the invention, it is not mandatory to produce the polypeptide or the fusion protein of the invention in a eukaryotic context that will favour post-translational modifications (e.g. glycosylation). Furthermore, prokaryotic cells have the advantages to produce protein in large amounts. If a eukaryotic context is needed, yeasts (e.g. saccharomyces strains) may be particularly suitable since they allow production of large amounts of proteins. Otherwise, typical eukaryotic cell lines such as CHO, BHK-21, COS-7, C127, PER.C6, YB2/0, HEK293, mononuclear macrophage/monocyte-lineage hematopoietic precursors, Haematopoietic stem cells, Mononuclear precursor cells, osteoblast or inactive osteoclast could be used, for their ability to process to the right post-translational modifications of the fusion protein of the invention.

The construction of expression vectors in accordance with the invention, and the transformation of the host cells can be carried out using conventional molecular biology techniques. The protein, peptide or the fusion protein of the invention, can, for example, be obtained by culturing genetically transformed cells in accordance with the invention and recovering the polypeptide or the fusion protein expressed by said cell, from the culture. They may then, if necessary, be purified by conventional procedures, known in themselves to those skilled in the art, for example by fractional precipitation, in particular ammonium sulfate precipitation, electrophoresis, gel filtration, affinity chromatography, etc. In particular, conventional methods for preparing and purifying recombinant proteins may be used for producing the proteins in accordance with the invention.

In a particular embodiment, the agent for use for increasing PAX6 gene and/or protein expression is a small molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the agent for use for increasing PAX6 gene and/or protein expression is duloxetine, ritanserin, topotecan and/or their derivatives.

In a particular embodiment, the agent for use for increasing PAX6 gene and/or protein expression is topotecan, duloxetine and/or its derivatives. Duloxetine is a Serotonin and Norepinephrine Reuptake Inhibitor. The mechanism of action of duloxetine is as a Norepinephrine Uptake Inhibitor, and Serotonin Uptake Inhibitor. Duloxetine is marketed as duloxetine Hydrochloride under the trade name Cymbalta® by Eli Lilly. Following patent applications describe duloxetine, its derivatives and different formulations: U.S. Pat. Nos. 5,023,269; 5,508,276; U.S. Patent Application No. 20060079569; No. 20070004795; No. 20060182796; No. 20070149479; International (PCT) Application Publication No. WO2007034503A2. Duloxetine has the following formula and structure in the art: CAS numbers: 116817-13-1 (racemic); 116539-59-4 S(+); 116539-60-7 R(−).

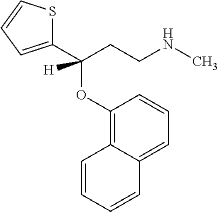 [SMILES: CNCC[C@H](Oc1cccc2ccccc12)c1cccs1]

In particular embodiment, the agent for use for increasing PAX6 gene and/or protein expression is a duloxetine derivative having the following formula:

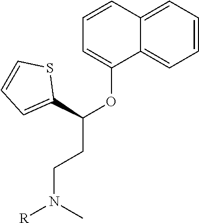

wherein —R is selected in the group consisting in: -D, —CH₃, —CH₂—(CH₃)₂, —CO—CH₂—CH₃ or —CO—(CH₂)₂—O—(CH₂)₂—O—CH₃.

In a particular embodiment, the agent for use for increasing PAX6 gene and/or protein expression is ritanserin. Ritanserin is a serotonin receptor antagonist. Ritanserin has the CAS Number 87051-43-2 and the following structure in the art:

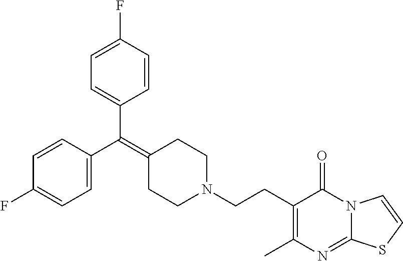 [SMILES: Cc1nc2sccn2c(=O)c1CCN1CCC(=C(c2ccc(F)cc2)c2ccc(F)cc2)CC1]

In particular embodiment, the agent for use for increasing PAX6 gene and/or protein expression is a ritanserin derivative having the following formula:

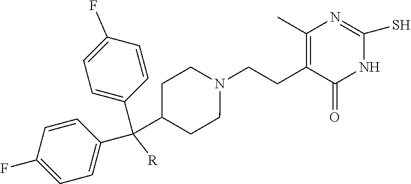

wherein —R is selected in the group consisting in: —NH—CH₃, N—(CH₃)₂, —NH—CO—CH₂—CH₃ or —NH—CO—(CH₂)₂—O—(CH₂)₂—O—CH₃

In another embodiment, the agent for use for increasing PAX6 gene and/or protein expression is topotecan. It is topoisomerase inhibitor and used as a chemotherapeutic agent. Topotecan has the trade name as Hycamti. Topotecan and its derivatives are described in U.S. Pat. No. 5,004,758. Topotecan has the CAS Number 123948-87-8 and the following structure in the art:

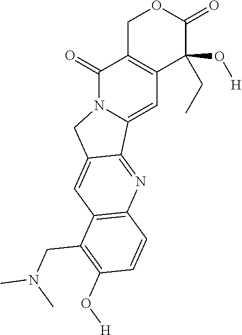 [SMILES: CC[C@@]1(O)C(=O)OCc2c1cc1n(c2=O)Cc2cc3c(CN(C)C)c(O)ccc3nc2-1]

In particular embodiment, the agent for use for increasing PAX6 gene and/or protein expression is a ritanserin derivative having the following formula:

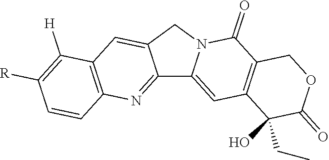

wherein —R is selected in the group consisting in: —OH, —NO₂ or —H.

In particular embodiment, the agent for use for increasing PAX6 gene and/or protein expression is a ritanserin derivative having the following formula:

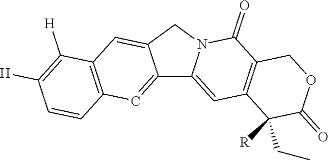

wherein —R is —OH or —O—CH₃.

In particular embodiment, the agent for use for increasing PAX6 gene and/or protein expression is a ritanserin derivative having the following formula:

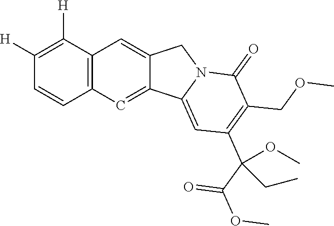 [SMILES: CCC(OC)(C(=O)OC)c1cc2n(c(=O)c1COC)Cc1cc3ccccc3cc1-2]

In particular embodiment, the agent for use for increasing PAX6 gene and/or protein expression is a ritanserin derivative having the following formula:

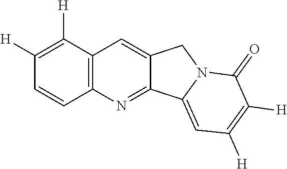 [SMILES: O=c1cccc2n1Cc1cc3ccccc3nc1-2]

In particular embodiment, the agent for use for increasing PAX6 gene and/or protein expression is a ritanserin derivative having the following formula:

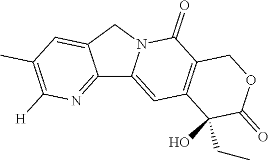 [SMILES: CC[C@@]1(O)C(=O)OCc2c1cc1n(c2=O)Cc2cc(C)cnc2-1]

In particular embodiment, the agent for use for increasing PAX6 gene and/or protein expression is a ritanserin derivative having the following formula:

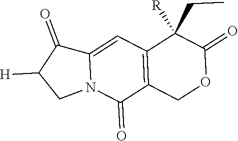

wherein —R is —OH or —O—CH₃.

In a second aspect, the invention relates to a i) duloxetine, ii) ritanserin, iii) topotecan and/or their derivatives, as a combined preparation for simultaneous, separate or sequential use in the method for increasing PAX6 gene and/or protein expression in a subject in need thereof.

The agent for increasing PAX6 protein expression according to the invention, wherein duloxetine, ritanserin, topotecan and/or their derivatives are used as a combined preparation.

More particularly, the invention relates to a i) duloxetine, ii) ritanserin, iii) topotecan and/or their derivatives, as a combined preparation for simultaneous, separate or sequential use in the treatment of PAX6-related deficiencies diseases.

As used herein the terms “administering” or “administration” refer to the act of injecting or otherwise physically delivering a substance as it exists outside the body (e.g. Duloxetine, ii) Ritanserin, iii) Topotecan and/or their derivatives) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In a particular embodiment, the subject is administered orally, subcutaneously, intradermally or topically with an agent which increases PAX6 protein expression (e.g., duloxetine, ritanserin, topotecan and/or their derivatives). In a further embodiment, the method according to the invention, wherein duloxetine, ritanserin, topotecan and/or their derivatives are administered to the subject in need thereof simultaneously, separately or sequentially.

As used herein, the term “administration simultaneously” refers to administration of at least 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In a particular embodiment, the agent is formulated as an oral, subcutaneous, intradermal, ocular or topical formulation.

In a particular embodiment, duloxetine, ritanserin, topotecan and/or their derivatives are administered by an oral, subcutaneous, intradermal, ocular or topical administration.

In a particular embodiment, the administration of topotecan is performed by a topical administration. In a particular embodiment, the administration of topotecan is performed by an intravitreal administration. More particularly, topotecan is formulated as a cream, an ointment, or gel. In another embodiment, the topotecan is formulated as an ophthalmic drop or an ophthalmic ointment.

In a particular embodiment, the administration of ritanserin is performed by an oral or intravenous administration. In a particular embodiment, the administration of duloxetine is performed by an oral or intravenous administration.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Pharmaceutical Composition

In a third aspect, the invention relates to a pharmaceutical composition comprising an agent which increases PAX6 gene and/or protein expression. The pharmaceutical composition according the invention, wherein the agent is duloxetine, ritanserin, topotecan and/or their derivatives.

More particularly, the pharmaceutical composition according to the invention is suitable for treating PAX6-related deficiencies disease.

In a particular embodiment, the pharmaceutical composition according the invention comprising duloxetine, ritanserin, topotecan and/or their derivatives as a combined preparation for simultaneous, separate or sequential use in the treatment of PAX6-related deficiencies diseases.

The pharmaceutical composition according the invention for use in the treatment of aniridia and/or diabetes in a subject in need thereof.

The agent which increases PAX6 gene and/or protein expression as defined above and the pharmaceutical combination according to the invention, as described above may be combined with pharmaceutically acceptable excipients, and optionally sustained-release matrices, such as biodegradable polymers, to form pharmaceutical compositions.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In some embodiments, the pharmaceutical formulation can be suitable orally, subcutaneously, intradermally, ocularly or topically administration.

In some embodiments, the pharmaceutical formulation can be suitable for topical administration.

More particularly, the pharmaceutical composition comprising topotecan is formulated for a topical administration. In a particular embodiment, pharmaceutical composition comprising topotecan is formulated for an intravitreal administration. In another embodiment, pharmaceutical composition comprising topotecan is formulated as a cream, an ointment, or gel. In another embodiment, the pharmaceutical composition comprising topotecan is formulated as an ophthalmic drop or an ophthalmic ointment.

In another embodiment, the pharmaceutical composition comprising ritanserin is formulated for an oral or intravenous administration.

In another embodiment, the pharmaceutical composition comprising duloxetine is performed by an oral or intravenous administration.

In certain embodiments, the present invention provides a topical formulation comprising duloxetine, ritanserin, topotecan and/or their derivatives.

Dosage forms for the topical or transdermal administration of the inhibitors of the present invention include, but are not limited to, powders, sprays, ointments, pastes, creams, lotions, gels, solutions, patches and inhalants. In certain non-limiting embodiments, a topical formulation comprises duloxetine, ritanserin, topotecan and/or their derivatives comprised in micelles, liposomes, or non-lipid based microspheres. In certain non-limiting embodiments, such a topical formulation may comprise a permeability enhancing agent such as but not limited to dimethyl sulfoxide, hydrocarbons (for example, alkanes and alkenes), alcohols (for example, glycols and glycerols), acids (for example, fatty acids), amines, amides, esters (for example, isopropyl myristate), surfactants (for example, anionic, cationic, or non-ionic surfactants), terpenes, and lipids (for example, phospholipids).

In certain embodiments, the pharmaceutical formulation can be suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In some embodiments, the present invention provides a parenteral formulation comprising duloxetine, ritanserin, topotecan and/or their derivatives as a combined preparation.

Method of Screening

A further object of the present invention relates to a method of screening a drug suitable for the treatment of PAX6-related deficiencies disease comprising i) providing a test compound and ii) determining the ability of said test compound to activate the activity and/or expression of PAX6.

Any biological assay well known in the art could be suitable for determining the ability of the test compound to activate and/or increase the activity and/or expression of PAX6. In some embodiments, the assay first comprises determining the ability of the test compound to bind to PAX6. In some embodiments, a population of cells is then contacted and activated so as to determine the ability of the test compound to activate the activity of PAX6. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of activating the activity of PAX6, as determined using in vitro methods described herein, are likely to exhibit similar modulatory capacity in applications in vivo. Typically, the test compound is selected from the group consisting of peptides, peptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: Topotecan can rescue PAX6 haploinsufficiency in vitro. Mutated (mut-LT) imbal stem cells were treated (dark blue) with 0.5 mM topotecan or DMSO (light blue) for 10 h and compared to normal limbal cells treated with DMSO (LT, white) by either qRT-PCR for PAX6-target gene expression (A) or western blot analysis for PAX6 protein production (B). The data illustrate several independent experiments (n=5 for A and n=3 for B). Oneway ANOVA followed by Dunnett's test was performed *p<0.05, **p<0.01.

FIG. 2: Topotecan can rescue migration of mutated limbal cells. Normal LT (blue) and mut-LT (orange and red) were tested for migration rate on IBIDI chambers. Mut-LT were treated (red) or not (orange) with 0.5 mM topotecan 2 h before migration start. DMSO was used as control. Numbers represent the percentage of remaining open area. n=2

FIG. 3: Topotecan activates the WT allele transcription in mutated cells but not in normal LSCs. qRT-PCR analysis for PAX6 expression was done on WT and mut-LSC+/− topotecan. Amplicons of mut-LSCs+Topotecan was sequenced and did not identified any amplification of the mutated allele. It suggests that topotecan does not act as forced read through the stop codon (like ataluren). Of interest, PAX6 in treated WT-LSCs is not enhanced, suggesting a fine regulation like in vivo.

FIG. 4: Ritanserine rescued PAX6 protein production. (A). Mutated LT (W) were treated (grey) or not (orange) with Ritanserine (1 mM) for 16 h and cell proliferation was measured and compared to untreated WT limbal cells (LT; blue). PAX6-target gene expression was analyzed by qRT-PCR. (B). Representative (over 3) western blot analyses on LT and W cells untreated and treated with MetOH buffer (buf) or Ritanserine (Rit) at 0.5, 1 or 5 mM.

FIG. 5: Duloxetine rescued PAX6 protein production. (A) qRT-PCR analysis of PAX6-target gene expression on WT control limbal cells (blue), or W mutant limbal cells (grery and orange) following treatment with buffer (grey) or 1 mM of Duloxetine (orange). (B) The data illustrate several independent experiments (n=5 for A and n=3 for B). One way ANOVA followed by Duneett's test was performed *p<0.05, **p<0.01. (C). Normal LSC (blue) and mut-LSC (orange and grey) were used as control for migration rate on IBIDI chambers. Mut-LSC were treated (orange) or not (grey) with 0.5 μM duloxetine 2 h before migration start. DMSO was used as control. Numbers represent the percentage of remaining open area; n=2.

### EXAMPLE

Material & Methods

Cells and culture conditions: Human mutated limbal epithelial stem cells (mut-LSCs) were obtained by genome editing (Crispr/cas9) of primary limbal stem cells, as described in Roux et al. (Roux, 2018 Stem cells). mut-LSCs and WT controls were cultured, as described earlier (Roux, 2018 Stem cells), in Keratinocyte serum-free medium (K-sfm) (Gibco™, Life Technologies), supplemented with 25 μg/mL Bovine Pituitary Extract (BPE; Gibco™, Life Technologies), 0.2 ng/mL Epidermal Growth Factor (EGF, Peprotech), 0.4 mM CaCl₂, 2 mM Glutamine (Gibco™, Life Technologies) and 100 U/mL Penicillin/Streptomicin (Gibco™, Life Technologies). Routine subcultures were obtained by detaching cells with StemPro™ Accutase™ Cell Dissociation Reagent (Gibco™, Life Technologies) and replating at 2,000 cells/cm² (T-LSCs) or 3,000 cells/cm² (PAX6^{+/-} T-LSCs). All cells were negative for mycoplasma contamination (monthly tested).

Rescue experiments: Mut-LSCs were treated with each compound (Duloxetine, Ritanserine and Topotecan) directly added into the culture medium from 0.5 μg/mL to 4 μg/mL. No cell toxicity has been observed up to 10 μg/mL. The optimal concentration for each experiment (qRT-PCR, cell migration and cell adhesion) was 0.5 μg/mL. As positive control, 2 μg/mL recombinant PAX6 protein coupled with an 11R-tag (recPAX6, LD Biopharma Inc) was added, as described elsewhere (Roux et al. 2018 Stem cells).

qRT-PCR analysis: Cells were treated for 10 h with either compound or recPAX6 for rescue experiments, and harvested as a dry pellet. RNA was then extracted using RNEasy Mini kit (Qiagen) and cDNA were synthetized from 1 μg RNA using iScript cDNA synthesis kit (Bio-Rad). Quantitative PCR were performed in triplicate using 2λ SYBR Green PCR Master Mix (Absource Biotools). Expression of each gene was calculated using the 2^{-ΔΔCt} method. Results are presented as fold change normalized to B2M house-keeping gene and relative to control (treated with protein buffer or untreated) T-LSCs. Specific primers sequences used are listed in Suppl. Table I.

Western blot analysis: Cells were harvested as a dry pellet, after 16 h or 24 h of treatment with each compound or 2 μg/mL recPAX6 protein or its buffer for rescue experiments, lysed in RIPA buffer supplemented with Protease and Phosphatase Inhibitor Cocktail EDTA-Free (Roche) for 15 min on ice and centrifuged for 15 min at 4° C. at 15,000×g. Protein concentration was measured using Pierce BCA Protein Assay kit (Thermo Fisher Scientific) following manufacturer instructions. Thirty micrograms of total protein or 10 ng of recPAX6 protein were loaded on a SDS-PAGE gel (10%), transferred to nitrocellulose membranes using semi-dry method. Membranes were pre-stripped using Re-Blot Plus Strong solution (EMD Millipore) for 15 min, blocked twice for 5 min in 5% milk solution and incubated in primary antibody (PAX6, ab2237, Millipore, 1/1,000) overnight at 4° C. After 3 washing of 10 min in TBS with 0.2% Tween (TBS-T), membranes were incubated for 1 h with secondary antibody (Goat anti-rabbit HRP, BD Pharmingen) diluted in TBS-T 5% milk solution at 1/1,000. Proteins were visualized using chemiluminescence detection (Clarity™ Western ECL Substrate, Bio-Rad) on a gel imaging system (ImageQuant LAS 4000). Membranes were stripped again, incubated 90 min at room temperature with ACTIN (sc1615, 1/500, Santa Cruz) antibody diluted in TBS-T 5% milk, washed 3×10 min in TBS-T and incubated for 1 h in secondary antibody (Rabbit anti-goat HRP, 1/20,000, Jackson ImmunoResearch) at room temperature before revelation. Signal quantifications were made using ImageJ 1.49 software (NIH). Results are presented after ACTIN normalization.

In vitro migration test: WT and mut-LSCs were seeded into culture-inserts 2 well at 25,000 cells per well in 6-well plates (IBIDI®). After adhesion, cells were treated with each compound at 0.5 μg/mL or recPAX6 as positive control for rescue experiments. Sixteen hours after plating, the inserts were removed creating a gap of 500 μm between cells. Patches were overlaid with culture medium and closure of the gap was monitored for 10 h, taking pictures at regular time intervals. Gap width was measured using ImageJ 1.49 software (NIH). Results are presented as percent of gap width normalized at 100% at t=0 h.

Statistical analysis: Data are expressed as means +/−SEM except for Gene Ontology charts as indicated and analyzed by Prism v7.04 (GraphPad software, Inc). Normality was first evaluated using Shapiro-Wilk test. Then, unpaired t-test (WT-LSCs vs mut-LSCs experiments), one-way ANOVA followed by multiple comparison Dunnett's test (rescue experiments, proliferation and WB figures) or two-way ANOVA followed by Bonferroni's test (migration assays) were performed, as indicated in legends, to calculate p-values. Differences are considered to be statistically significant from a p-value below 0.05.

Results:

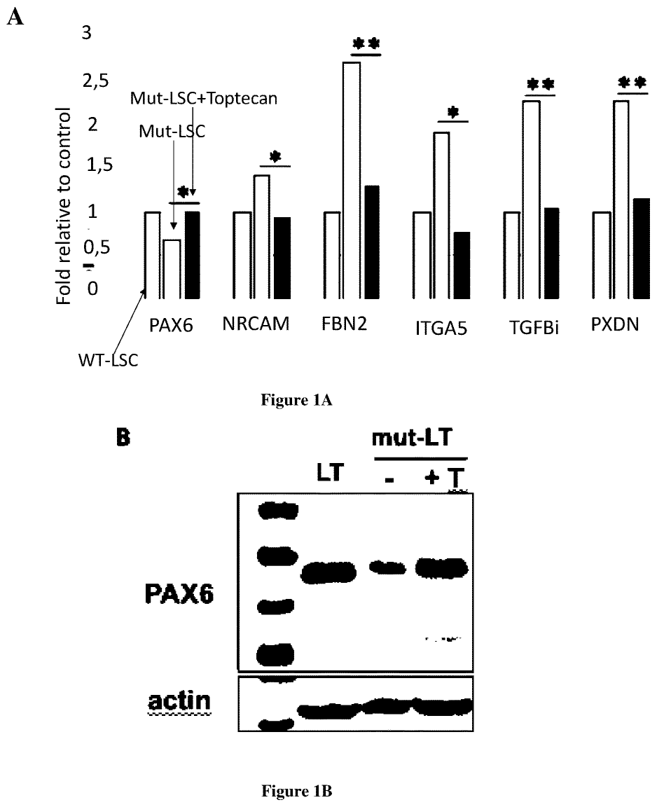

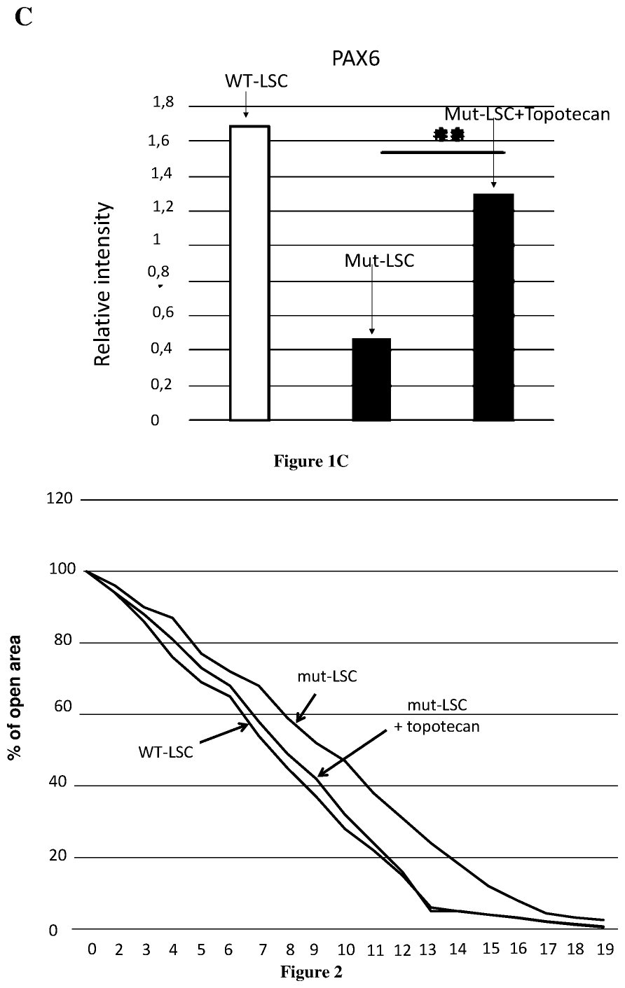

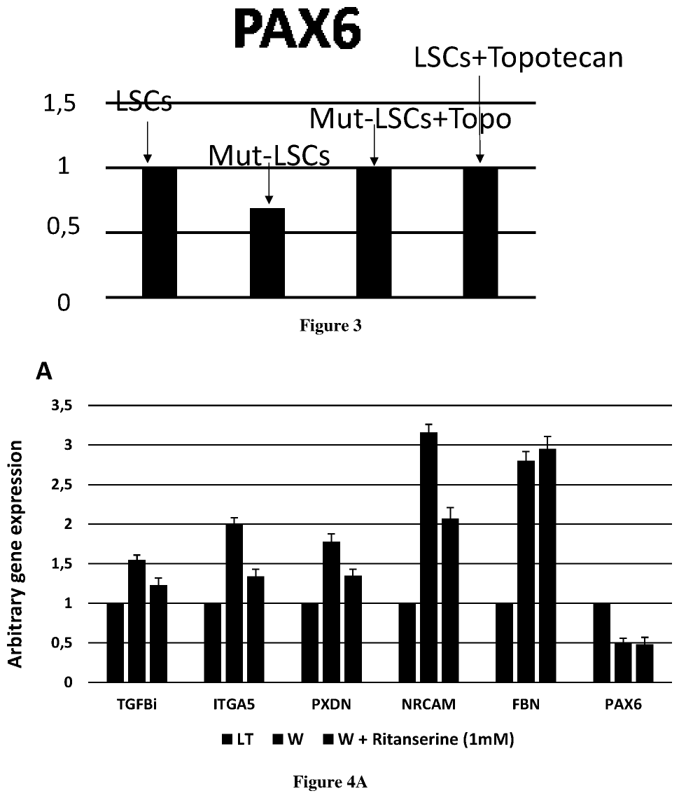

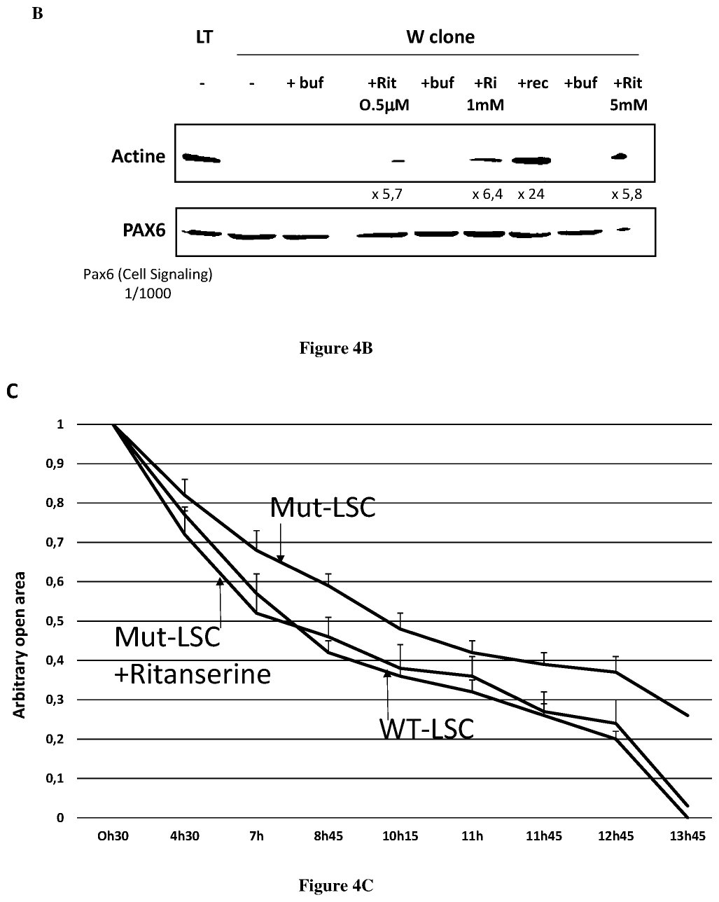

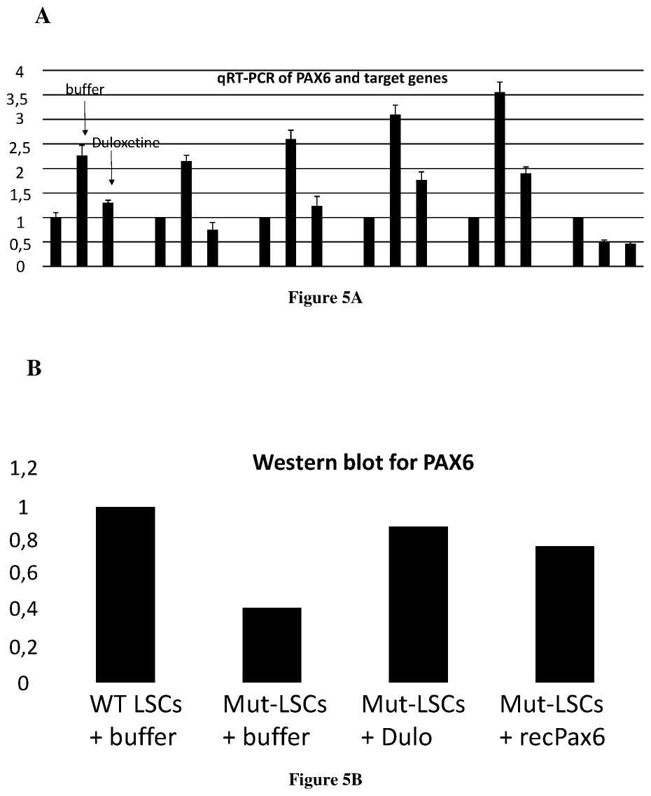

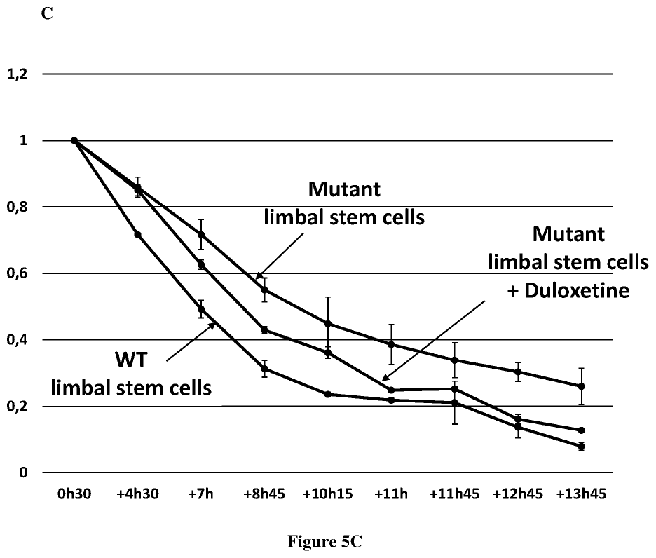

As illustrated on FIGS. 1 to 5, each of the three compounds: Topotecan (FIGS. 1 to 3), Ritanserine (FIG. 4) or Duloxetine (FIG. 5) is able to rescue the production of endogenous PAX6 protein, PAX6-target gene expression and cell migration of mutated limbal stem cells, comparable to the rescue effect of recombinant PAX6 protein treatment, as described in Roux et al. (Roux 2018 Stem cells). Topotecan is able to rescue PAX6 gene expression at the RNA level. It suggests that duloxetine or ritanserine stabilizes PAX6 protein while Topotecan enhances its transcription.

### REFERENCES

Throughout this application, various references describe the state of the art to which this invention pertains. The disclosures of these references are hereby incorporated by reference into the present disclosure.
1. Landsend E S, Utheim Ø A, Pedersen H R, Lagali N, Baraas R C, Utheim T P. The Genetics of Congenital Aniridia—A Guide for the Ophthalmologist. Surv Ophthalmol 2018; 63:105-113.
2. Baulmann D C, Ohlmann A, Flügel-Koch C, Goswami S, Cvekl A, Tamm E R. (2002) Pax6 heterozygous eyes show defects in chamber angle differentiation that are associated with a wide spectrum of other anterior eye segment abnormalities. Mech Dev. 118(1-2):3-17.
3. Koroma B M, Yang J M, Sundin O H. (1997) The Pax-6 homeobox gene is expressed throughout the corneal and conjunctival epithelia. Invest Ophthalmol Vis Sci. 38(1):108-20.
4. Shaham O, Menuchin Y, Farhy C, Ashery-Padan R. (2012) Pax6: a multi-level regulator of ocular development. Prog Retin Eye Res. 31(5):351-76.
5. Pellegrini G, Rama P, Di Rocco A, Panaras A, De Luca M. (2014) Concise review: hurdles in a successful example of limbal stem cell-based regenerative medicine. Stem Cells. 32(1):26-34.
6. Ihnatko R, Eden U, Fagerholm P, Lagali N. Congenital Aniridia and the Ocular Surface. Ocul Surf. 2016 April; 14(2):196-206.
7. Parekh M, Poli B, Ferrari S, Teofili C, Ponzin D Aniridia: Recent Developments in Scientific and Clinical Research. (Eds.). Springer International, Switzerland, 2015. ISBN: 978-3-319-19778-4.8. Vicente, A., Bystrom, B. Pedrosa Domellof, F. (2018) Altered Signaling Pathways in Aniridia-Related Keratopathy. Invest Ophthalmol Vis Sci, 59(13): 5531-41.
9. Strungaru M H, Mah D, Chan C C. Focal limbal stem cell deficiency in Turner syndrome: report of two patients and review of the literature. Cornea 2014; 33(2):207-9.
10. Skeens H M, Brooks B P, Holland E J. Congenital aniridia variant: minimally abnormal irides with severe limbal stem cell deficiency. Ophthalmology 2011; 118(7):1260-4.
11. Hatch K M, Dana R. The structure and function of the limbal stem cell and the disease states associated with limbal stem cell deficiency. International ophthalmology clinics 2009; 49(1):43-52.
12. Di Iorio E, Kaye S B, Ponzin D, et al. Limbal stem cell deficiency and ocular phenotype in ectrodactyly-ectodermal dysplasia-clefting syndrome caused by p63 mutations. Ophthalmology 2012; 119(1): 74-83.
13. Messmer E M, Kenyon K R, Rittinger O, Janecke A R, Kampik A. Ocular manifestations of keratitis-ichthyosis-deafness (KID) syndrome. Ophthalmology 2005; 112(2):e1-6.
14. Fernandes M, Sangwan V S, Vemuganti G K. Limbal stem cell deficiency and xeroderma pigmentosum: a case report. Eye 2004; 18(7):741-3.
15. Lim P, Fuchsluger T A, Jurkunas U V. Limbal stem cell deficiency and corneal neovascularization. Seminars in ophthalmology 2009; 24(3):139-48.
16. Aslan D, Ozdek S, Camurdan O, Bideci A, Cinaz P. Dyskeratosis congenita with corneal limbal insufficiency. Pediatric blood & cancer 2009; 53(1):95-7.
17. Gosmain Y, Katz L S, Masson M H, Cheyssac C, Poisson C, Philippe J. Pax6 is crucial for β-cell function, insulin biosynthesis, and glucose-induced insulin secretion. Mol

Endocrinol. 2012 April; 26(4):696-709.
18. Swisa A, Avrahami D, Eden N, Zhang J, Feleke E, Dahan T, Cohen-Tayar Y, Stolovich-Rain M, Kaestner K H, Glaser B, Ashery-Padan R, Dor Y. PAX6 maintains β cell identity by repressing genes of alternative islet cell types. J Clin Invest. 2017 January 3; 127(1):230-243.
19. Ding J, Gao Y, Zhao J, Yan H, Guo S Y, Zhang Q X, Li L S, Gao X. Pax6 haploinsufficiency causes abnormal metabolic homeostasis by down-regulating glucagon-like peptide 1 in mice. Endocrinology. 2009 May; 150(5):2136-44.
20. Yasuda T, Kajimoto Y, Fujitani Y, Watada H, Yamamoto S, Watarai T, Umayahara Y, Matsuhisa M, Gorogawa S, Kuwayama Y, Tano Y, Yamasaki Y, Hori M 2002 PAX6 mutation as a genetic factor common to aniridia and glucose intolerance. Diabetes 51:224-230
21. Laakso M. Not for the eyes only: PAX6 and glucose metabolism. Diabetologia 2009 March; 52:381-4.
22. Roux L N, Petit I, Domart R, Concordet J P, Qu J, Zhou H, Joliot A, Ferrigno O, Aberdam D. Modeling of Aniridia-Related Keratopathy by CRISPR/Cas9 Genome Editing of Human Limbal Epithelial Cells and Rescue by Recombinant PAX6 Protein. Stem Cells. 2018 36(9): 1421-1429.
23. Ghassemi F, Shields C L, Ghadimi H, Khodabandeh A, Roohipoor R. Combined intravitreal melphalan and topotecan for refractory or recurrent vitreous seeding from retinoblastoma. JAMA Ophthalmol. 2014 August; 132(8):936-41.
24. Chen Y, Feng R, Wang H, Wei R, Yang J, Wang L, Wang H, Zhang L, Hong T P, Wen J. High-fat diet induces early-onset diabetes in heterozygous Pax6 mutant mice. Diabetes Metab Res Rev. 2014 September; 30(6):467-75.
25. Manjandavida F P, Shields C L. The role of intravitreal chemotherapy for retinoblastoma. Indian J Ophthalmol. 2015 February; 63(2):141-5. doi: 10.4103/0301-4738.154390. Review.

1-7. (canceled)

8. A pharmaceutical composition comprising an agent which increases PAX6 gene and/or protein expression.

9. The pharmaceutical composition according to claim 8, wherein said agent is duloxetine, ritanserin, topotecan and/or a derivative thereof.

10. (canceled)

11. A method for treating a PAX6-related deficiency diseases in a subject need thereof comprising a step of administering to said subject a therapeutically effective amount of an agent which increases PAX6 gene and/or protein expression.

12. The method of claim 11, wherein the PAX6-related deficiency disease is aniridia and/or diabetes.

13. The method of claim 11, wherein the agent is an aptamer, a small molecule, an antibody, a peptide, a polypeptide peptidomimetic or glycomimetic.

14. The method of claim 11, wherein the agent is duloxetine, ritanserin, topotecan and/or a derivative thereof.

15. The method of claim 14, wherein the duloxetine, the ritanserin, the topotecan and/or the derivative is/are formulated in a combined preparation.

16. The method of claim 11, wherein said agent is formulated for oral, cutaneous, topical or ocular use.
